(19) United States
(12) Patent Application Publication
(10) Pub. No.: US 2014/0322148 A1
(43) Pub. Date: Oct. 30, 2014

(54) Oil Compositions and Methods for Increasing Hair Growth and/or Preventing Hair Loss

(71) Applicant: Adrianna Janell JACKSON, Houston, TX (US)

(72) Inventor: Adrianna Janell Jackson, Houston, TX (US)

(21) Appl. No.: 14/360,302

(22) PCT Filed: Nov. 20, 2012
(86) PCT No.: PCT/US12/66166

§ 371 Date: May 22, 2014

Related U.S. Application Data
(60) Provisional application No. 61/563,605, filed on Nov. 25, 2011.

(51) Int. Cl. A61K 8/63; A61K 8/42; A61K 8/92; A61Q 7/00; A61Q 7/02; A61K 8/49; A61K 8/36

(52) U.S. Cl. CPC A61K 8/63; A61K 8/4953; A61K 8/42; A61K 8/361; A61Q 7/00; A61Q 7/02; A61K 8/922; A61K 8/925; A61K 8/92
USPC 424/59; 514/275; 424/718; 514/622; 514/530; 514/573; 514/570; 514/559; 514/284; 514/569; 514/337

(57) ABSTRACT

Described herein are topical oil-based formulations that find use in the treatment of alopecia, i.e., hair loss, and in the promotion of hair growth.

### TECHNICAL FIELD

This invention relates to methods for oil-based compositions and methods for treating hair loss. The composition is applied daily to the affected area, e.g., the scalp. More particularly, this invention relates to compositions and methods for arresting or reversing hair loss, or both, and promoting hair growth.

### BACKGROUND

Dermatologists recognize many different types of hair loss, the most common by far being “androgenetic alopecia” wherein human males or females begin losing scalp hair at the temples and on the crown of the head. While this type of hair loss is largely confined to males, hence its common name “male pattern baldness,” it is not unknown in women.

Androgenetic alopecia (AGA) is the most common form of hair loss, affecting approximately 50 percent of all men and women by the age of 50. Common treatments for androgenetic alopecia include hair follicle transplants, topical therapies, and orally prescribed anti-androgens. The hyperandrogenic stimulation that causes androgenetic alopecia also produces other undesirable physiological symptoms including acne vulgaris, benign prostatic hyperplasia, female hirsutism, and seborrhea.

Minoxidil (6-(1-piperidinyl)-2,4-pyrimidinediamide 3 oxide) is a drug known for the treatment of AGA. Minoxidil is effective when delivered topically at a concentration of about 0.01% to about 5%. The topical solution of Minoxidil is currently marketed as “Rogaine” having 2% minoxidil concentration in a solution of (60% v/v) propylene glycol, and water for women and 5% minoxidil concentration for men.

Finasteride is a synthetic androgen inhibitor currently marketed as Proscar® for the treatment of benign prostatic hyperplasia. It has also been marketed orally in low dosage form as Propecia® for the treatment of male androgenetic alopecia (hair loss). Chemically, finasteride is (5α, 17β)-N-(1,1-dimethylethyl)-3-oxo-4-azaandrost-1-ene-17-carboxamide and is practically insoluble in water, and is soluble only in organic solvents such as ethanol and methanol. Finasteride is known to inhibit 5-alpha-reductase type 2, which inhibits testosterone's conversion to dihydrotestosterone (DHT). In AGA susceptible scalp hair follicles, DHT initiates a cascade of events that lead to the gradual transformation of large terminal hair follicles to miniaturized hair follicles. Finasteride decreases serum and scalp DHT which leads to an increase in hair growth.

Although, finasteride is currently administered orally, it is not available topically as approved product for the treatment of AGA. The side effects as a result of the oral administration for finasteride include erectile dysfunction, impotence, low libido, decreased ejaculate, gynecomestica, and facial hair growth.

Inhibitors of testosterone-5α-reductase will serve to prevent or lessen symptoms of hyperandrogenetic stimulation in organs with endogenous 5-α dihydrotestosterone formation, e.g., the prostate (Gormley et al. U.S. Pat. No. 5,981,543, 1999 and Rasmusson et al. U.S. Pat. No. 4,377,584, 1983). Prostaglandin analogs have recently been further evaluated for their ability to promote hair growth, including eyelash growth/enhancement; see, e.g., Wolf, et al., 2003 Dermatology Online Journal 9(3): 7, and references cited therein.

Prior compositions used for arresting or reversing hair loss, and/or promoting hair growth are formulations with a high content of water and/or alcohol. Use of these prior compositions by people with curly/kinky hair interfere with styling (e.g., chemically or thermally straightened hair). The high water content of some current formulations reverse straight hair styles to a curly texture or cause frizzing. Formulations that are alcohol based will dry kinky/curly hair. Thus, there is a need for a person with naturally curly or kinky hair for a composition that does not interfere with styling or dry out the hair.

The presently described compositions and methods of use provide an oil-based composition that does not interfere with styling of naturally curly or kinky hair or drying out the hair.

### BRIEF SUMMARY OF THE INVENTION

Conventional topical hair growth, e.g., minoxidil, formulations consist of propylene glycol-water-ethanol solutions. However, twice daily applications of these formulations can cause severe adverse reactions, such as scalp irritation, burning, dryness, redness, allergic contact dermatitis, etc. Furthermore, the high water and/or alcohol content interfere with styling (e.g., chemically or thermally straightened hair) by either frizzing and/or drying out the hair. To minimize these side effects and to enhance therapeutic efficacy, formulations that provide the therapeutic agents in a manner that do not interfere with the styling of naturally curly or kinky hair are advantageous.

In a first aspect there is provided a topical oil-based pharmaceutical composition for slowing or stopping hair loss and/or promoting hair regrowth, said composition comprising:
(a) at least one therapeutic agent selected from a vasodilator, a prostaglandin analog, a 5-alpha-reductase inhibitor and a vitamin A analog;
(b) at least one oil;
(c) a viscosity agent;
(d) water, and
(e) alcohol,
   wherein said composition is propylene glycol-free and does not reverse the affect of chemically or thermally straightened hair.

In an aspect there is provided a topical oil-based pharmaceutical composition for slowing or stopping hair loss and/or promoting hair regrowth, said composition comprising:
(a) at least one therapeutic agent selected from a vasodilator, a prostaglandin analog, a 5-alpha-reductase inhibitor and a vitamin A analog;
(b) at least one oil;
(c) a viscosity agent;
(d) water; and
(e) alcohol,
   wherein said composition is substantially free of propylene glycol and does not reverse the affect of chemically or thermally straightened hair.

In an embodiment, the therapeutic agent is present in an amount of about 0.01% w/w to about 20% w/w when it is a vasodilator, about 0.01% w/w to about 20% w/w when it is a prostaglandin analog, about 0.01% w/w to about 20% w/w when it is a 5-alpha-reductase inhibitor and/or about 0.01% w/w to about 20% w/w when it is a vitamin A analog.

In a second embodiment, the at least one therapeutic agent is a combination of two, three or four therapeutic agents. The combination is selected from (1) a vasodilator and a prostaglandin analog, (2) a vasodilator and a 5-alpha-reductase inhibitor, (3) a vasodilator and a vitamin A analog, (4) a prostaglandin analog and a 5-alpha-reductase inhibitor, (5) a prostaglandin analog and a vitamin A analog, (6) a 5-alpha-reductase inhibitor and a vitamin A analog, (7) a vasodilator, a prostaglandin analog, and a 5-alpha-reductase inhibitor, (8) a vasodilator, a prostaglandin analog and a vitamin A analog, (9) a prostaglandin analog, a 5-alpha-reductase inhibitor and a vitamin A analog and (10) a vasodilator, a 5-alpha-reductase inhibitor and a vitamin A analog.

In some embodiments, the therapeutic agent is a vasodilator. The vasodilator may be minoxidil. The minoxidil may present in an amount of about 2% w/w to about 10% w/w.

In some embodiments, the therapeutic agent is a prostaglandin analog. The prostaglandin analog is selected from bimatoprost, latanoprost, travoprost, unoprostone, fluprostenol and tafluprost. The bimatoprost may be present in an amount of about 0.03% to about 5% by weight of the composition. The tafluprost may be present in an amount of about 0.0015% to about 5% by weight of the composition.

In some embodiments, the therapeutic agent is a 5-alpha-reductase inhibitor is selected from finasteride and dutasteride. The 5-alpha-reductase inhibitor is present in an amount of about 0.25% to about 5% by weight of the composition. The 5-alpha-reductase inhibitor may be finasteride, dutasteride or a combination thereof.

In some embodiments, the therapeutic agent is a vitamin A analog. The vitamin A analog may be tretinoin. The tretinoin may present in an amount of about 0.025% to about 0.1% by weight of the composition.

In some embodiments, the oil originates from vegetable, marine or animal sources or is a synthetic oil. The may be a saturated, unsaturated or polyunsaturated oil. The oil is selected from borage seed oil, syzigium aromaticum oil, hempseed oil, herring oil, cod-liver oil, salmon oil, flaxseed oil, wheat germ oil, evening primrose oils, almond oil, babassu oil, borage oil, black currant seed oil, canola oil, castor oil, coconut oil, corn oil, cottonseed oil, emu oil, evening primrose oil, flax seed oil, grapeseed oil, groundnut oil (e.g., peanut), lanolin oil, linseed oil, mink oil, mustard seed oil, olive oil, palm oil, palm kernel oil, rapeseed oil, safflower oil, sesame oil, shark liver oil, silicon oil, soybean oil, sunflower oil, tree nut oil (e.g., almond, cashew, macadamia, walnut, hazelnut, pistachio, Brazil nuts, etc.), hydrogenated castor oil, hydrogenated coconut oil, hydrogenated palm oil, hydrogenated soybean oil, hydrogenated vegetable oil, a mixture of hydrogenated cottonseed oil and hydrogenated castor oil, partially hydrogenated soybean oil, a mixture of partially hydrogenated soybean oil and partially hydrogenated cottonseed oil, glyceryl trioleate, glyceryl trilinoleate, glyceryl trilinolenate, citrate thisocetyl triglyceride having 10-18 carbon atoms, a Ω3 polyunsaturated fatty acid triglyceride containing oil, omega-3 oil, omega-6 oil, and any combination thereof. The oil may be present in an amount of between about 50% w/w to about 99% w/w.

In some embodiments, the viscosity agent is a carbomer. The viscosity agent may be present in an amount of between about 0.1% w/w to about 50% w/w.

In all embodiments, the composition may further comprise pharmaceutically acceptable additives and ingredients selected from the group consisting of hair conditioners, panthenol derivatives, calcium pantothenate, colorants, fragrances, fragrance modifiers, vitamin E, penetration modifiers, surfactants, cosmetic agents, fatty acids and fatty acid esters, herbal extracts, henna, wetting agents, sunscreens, and anti-irritants.

In some embodiments, the water is present in an amount of less than about 30% w/w of the final composition.

In some embodiments, the alcohol present in an amount of between 0.1% w/w and about 50% w/w of the final composition. Preferably, the alcohol is less than 30% w/w, 20% w/w, 10% w/w or 5% w/w.

In some embodiments, the composition is an oil, cream, ointment, emulsion, gel, or lotion.

In some embodiments, the propylene glycol content the composition is less than about 10% w/w, preferably less than about 5% w/w.

In a second aspect, there is provided a method for treating or preventing hair loss in a region of a patient, said method comprising topically administering to said region a topical oil-based composition as described herein. The hair loss comprises androgenetic alopecia, frontal hair loss, bitemporal recession, vertex balding, mid-anterior balding, alopecia areata, anagen hair loss, diffuse alopecia, telogen effluvium, and traction alopecia.

In some embodiments, the composition is administered on the skin once or twice a day. The composition is administered for at least a week. In some embodiments, the composition is applied to the scalp.

In a third aspect, there is provided a method of preparing a topical oil-based composition, said method comprising, based on the weight of the final composition:
(a) from about 0.001% w/w to about 20% w/w of at least one therapeutic agent;
(b) from about 50% w/w to about 99% w/w of an oil;
(c) from about 10% w/w to about 50% w/w of an alcohol;
(d) from about 0.01% w/w to about 50% w/w of a viscosity agent; and
(e) from about 0% w/w to about 30% w/w water.
   wherein said therapeutic agent is substantially solubilized in said composition, said process comprising:
   (1) providing a solution comprising said therapeutic agent, said oil, and a portion of said alcohol;
   (2) providing a dispersion comprising said viscosity agent, the remaining portion of said alcohol, and said water; and
   (3) combining said solution and said dispersion to provide a final composition.

In one embodiment, there is provided a method for alleviating a condition characterized by inadequate or lack of hair, in or on a warm-blooded animal, e.g., a human, which comprises topically administering to said animal an effective amount of a pharmaceutical composition comprising: (1) at least one therapeutic agent and (2) an oil-based solution suitable for topical or other local application.

The compositions of the present invention further preferably comprise a pharmaceutically acceptable solvent, and in preferred form, the therapeutic agent may be substantially solubilized therein. In some embodiments it is specifically contemplated that the presently disclosed compositions do not contain petrolatum. In other embodiments, the petrolatum is present in an amount of less than about 20% of the final composition. In some embodiments, the composition may further comprise petrolatum in an amount less than about 20% w/w, less than about 15% w/w, less than about 10% w/w, or less than about 5% w/w. Further, in some embodiments, the compositions are not emulsions.

Other objects, features and advantages of the present invention will become apparent from the following detailed description. It should be understood, however, that the detailed description, while indicating preferred embodiments of the invention, are given by way of illustration only, since various changes and modifications within the scope and spirit of the invention will become apparent to one skilled in the art from this

### DETAILED DESCRIPTION

The invention will now be described in detail by way of reference only using the following definitions and examples. All patents and publications, including all disclosed within such patents and publications, referred to herein are expressly incorporated by reference.

Unless defined otherwise herein, all technical and scientific terms used herein have the same meaning as commonly understood by one of ordinary skill in the art to which this invention belongs. Numeric ranges are inclusive of the numbers defining the range.

The headings provided herein are not limitations of the various aspects or embodiments of the invention, which can be had by reference to the specification as a whole. Accordingly, the terms defined immediately below are more fully defined by reference to the specification as a whole.

### Definitions

As used herein, the term “administration” refers to the act of giving a drug, prodrug, or other active agent, or therapeutic treatment (e.g., compositions of the present invention) to a subject. Exemplary routes of administration to the human body can be on the skin, e.g., on the scalp. Administration may be in one or more administrations, applications or dosages, and is not intended to be limited to a particular time period. “Administered” and “applied” are used to describe the act of “administration” and are used synonymously.

As used herein, the terms “hair growth,” “regulating hair growth,” “hair growth regulation,” and “regulating the growth of hair,” are meant to include: stimulating hair growth; stimulating hair thickening; preventing, reducing, arresting and/or retarding the loss of hair; preventing, reducing, arresting and/or retarding the thinning of hair; increasing the rate of hair growth; inducing the formation of a greater number of hair strands; increasing the diameter of the hair strand; lengthening the hair strand; changing the hair follicle from a vellus or miniaturized follicle to a large terminal follicle; inducing the formation of vellus and terminal follicles.

The term “oil-based,” as used herein, refers to a composition in which the major component is an oil.

The term “pharmaceutically acceptable,” as used herein, refers to materials, which are generally not toxic, or injurious to a patient when used in the compositions of the present invention, including when the compositions are applied topically according to methods described herein.

The phrase “propylene glycol-free” means that a composition does not contain propylene glycol.

The term “substantially free” is meant that the component content in the composition is less than about 10% w/w. For example, a composition that is substantially free of petrolatum has a petrolatum content that is less than about 10% of the final composition.

The term “substantially solubilized,” as used herein, means that the therapeutic agent is dissolved in the compositions and is present at a concentration which is less than about its solubility limit in the present compositions.

The term “therapeutic agent,” as used herein, refers herein to a compound that is useful for the treatment, alleviation or prevention of hair loss, and/or the stimulation of hair growth. Exemplary agents are a hair growth stimulant, and/or hair growth stimulating agent and/or hair density increasing agent and/or hair loss prevention agent.

The term “topical” or “topically” in reference to administration or application of a composition or a product refers to epicutaneous administration or application, or administration onto the surface of the skin. The term “topically active agent” as used herein refers to a compound that is effective in a treatment of a skin condition when administered topically. It is to be understood that topically active agent can have a local or a systemic effect, or both, when administered topically. The term “topical,” when used in reference to a composition, formulation or a product refers to a composition or a product formulated for topical application.

The terms “vitamin A analog” and “retinoid” refer to a compound that is structurally similar to vitamin A. The terms “vitamin A analog” and “retinoid” are used interchangeably herein.

The terms “about” and “approximately equal” are used herein to modify a numerical value and indicate a defined range around that value. If “X” were the value, “about X” or “approximately equal to X” would generally indicate a value from 0.90X to 1.10X. Any reference to “about X” minimally indicates at least the values X, 0.90X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.10X. Thus, “about X” is intended to disclose, e.g., “0.98X.” When “about” is applied to the beginning of a numerical range, it applies to both ends of the range. Thus, “from about 6 to 8.5” is equivalent to “from about 6 to about 8.5.” When “about” is applied to the first value of a set of values, it applies to all values in that set. Thus, “about 7, 9, or 11%” is equivalent to “about 7%, about 9%, or about 11%.” “About” may also include variations in the amount that a regulatory body such as the FDA or EMEA would view as bioequivalent to the claimed amount.

### Hair Loss/Alopecia

There are many types of hair loss, commonly referred to as alopecia. The

Norwood classification system for male pattern hair loss is commonly used to evaluate the extent of hair loss. Hair loss is scored on the scale as follows:

Class I represents an adolescent or juvenile hairline and it not actually balding. The adolescent hairline generally rests on the upper brow crease.

Class II indicates a progression to the adult or mature hairline, which sits a finger breath (1.5 cm) above the upper brow crease, with some temporal recession. This also does not represent balding.

Class III is the earliest stage of male hair loss. It is characterized by a deepening temporal recession.

Class III Vertex represents early hair loss in the crown (vertex).

Class IV Is characterized by further frontal hair loss and enlargement of vertex, but there is still a solid band of hair across top separating front and vertex.

Class V the bald areas in the front and crown continue to enlarge and the bridge of hair separating the two areas begins to break down.

Class VI occurs when the connecting bridge of hair disappears leaving a single large bald area on the front and top of the scalp. The hair on the sides of the scalp remains relatively high.

Class VII patients have extensive hair loss with only a wreath of hair remaining in the back and sides of the scalp.

Class A patterns are less common than the regular pattern (<10%), but are significant because of the fact that, since the hair loss is most dramatic in the front, the patients look very bald even when the hair loss is minimal.

Women who suffer from Androgenetic Alopecia have a pattern of hair loss called female pattern hair loss. This loss does not occur in the same pattern as men but appears as a diffuse thinning throughout the scalp. There is a chart designed to classify female pattern hair loss called the Ludwig Scale, categorizing hair loss in women as Ludwig I, II or III depending on the severity of hair loss over the crown area.

Ludwig I specifies minimal widening of the central part width.

Ludwig II indicates moderate thinning of the central part.

Ludwig III represents significant tinning and widening of the central part width.

There are however always exceptions and in some cases men may suffer from a diffuse type thinning and women may experience a similar hair loss pattern to men.

The compositions provided for herein are for use in treating alopecia by alleviating (i.e., reducing the rate of hair loss) or preventing hair loss and/or enhancing hair growth. Specific forms of alopecia contemplated for treatment by the present compositions include, but are not limited to, androgenetic alopecia, non-scarring alopecia, frontal hair loss, bitemporal recession (e.g., Class III on the Norwood scale), vertex balding (e.g., Class III vertex on the Norwood scale), mid-anterior balding (Class A on the Norwood scale), alopecia areata, anagen hair loss, diffuse alopecia, telogen effluvium, and traction alopecia. A brief review of each is provided below but the listing should not be construed as limiting on the forms of hair loss that may be treated.

Androgenetic alopecia is a genetic condition that can affect both men and women. Men with this condition, called male pattern hair loss, male pattern baldness, or male pattern thinning can begin suffering hair loss as early as their teens or early 20s. It's characterized by a receding hairline and gradual disappearance of hair from the crown and frontal scalp. Women with this condition, called female pattern hair loss, female pattern baldness, or female pattern thinning, don't experience noticeable thinning until their 40s or later. Women experience a general thinning over the entire scalp, with the most extensive hair loss at the crown.

Alopecia areata often starts suddenly and causes patchy hair loss in children and adults. This condition may result in complete loss of all scalp hair (alopecia totalis) or loss of all scalp and body hair (alopecia universalis). But in about 90% of people with the condition, the hair returns within a few years.

Telogen effluvium is temporary hair thinning over the scalp that occurs because of changes in the growth cycle of hair. A large number of hairs enter the resting phase at the same time, causing hair shedding and subsequent thinning.

Age related thinning also known as senescent thinning, or involutional alopecia is a natural condition in which the hair gradually thins with age. More hair follicles go into the resting phase, and the remaining hairs become shorter and fewer in number.

Non-scarring alopecia refers to hair loss where the hair follicles are not permanently destroyed.

Frontal hair loss is a receding hairline either directly over the center of the forehead or at a man's temples. Frontal hair loss will start by the hair in these regions getting more fine and shorter and then falling out completely.

Traction alopecia is caused primarily by pulling force being applied to the hair. It is fairly unique to African-American females or individuals who wear tight hairstyles such as braids or ponytails.

Anagen hair loss occurs after any insult to the hair follicle that impairs its mitotic or metabolic activity. The hair loss is usually the result of an exposure to chemotherapeutic agents such as antimetabolites, alkylating agents, and mitotic inhibitors that are used to treat cancer, although it is not the only type of chemotherapy induced-hair loss in these patients.

Diffuse alopecia can affect both sexes at any age. It is characterized by thinning of the entire scalp, followed by thinning of the hair (the hair loses its density).

### Solvents

Exemplary alcohols include, for example, ethanol, propanol and butanol. Reference herein to “ethanol” includes absolute alcohol, as well as “alcohol USP” and all denatured forms of 95% ethanol. As used herein, the term “propanol” refers to all isomeric forms, including n-propanol and isopropanol, and the term “butanol” refers to all isomeric forms, including, for example, n-butanol, iso-butanol and sec-butanol. Preferred among these alcohols are ethanol and propanol, with ethanol being more preferred.

The amount of solvent employed in the compositions of the present invention may vary and will depend, for example, on the particular solvent and thickening agent employed, the therapeutic agent employed, and the like. Preferably, the solvent may be employed in a quantity sufficient to assure that the minoxidil is solubilized in the present compositions. Preferably, the solvent may be employed in the present compositions in an amount ranging from about 1% w/w to about 35% w/w, and all combinations and subcombinations of ranges and specific amounts therein. Preferably, when the solvent is solely an alcohol it may be employed in an amount of less than about 5% w/w, 5.5% w/w, 6% w/w, 6.5% w/w, 7% w/w, 7.5% w/w, 8% w/w, 8.5% w/w, 9% w/w, 9.5% w/w, 10% w/w, 10.5% w/w, 11% w/w, 11.5% w/w, 12% w/w, 12.5% w/w, 13% w/w, 13.5% w/w, 14% w/w, 14.5% w/w, 15% w/w, 15.5% w/w, 16% w/w, 16.5% w/w, 17% w/w, 17.5% w/w, 18% w/w, 18.5% w/w, 19% w/w, 19.5% w/w or 20% w/w of the final composition.

Even more preferably, the solvent(s) may be employed in the present compositions in an amount of from about 20% to about 99% by weight of the final composition, with concentrations of from about 30% to about 80% being yet more preferred.

### Oil Components & Concentrations

Suitable oils that find use in the presently disclosed compositions are oils that originate from vegetable, marine or animal sources or is a synthetic oil. The oil may be a saturated, unsaturated or polyunsaturated oil.

The oil is selected from borage seed oil, syzigium aromaticum oil, hempseed oil, herring oil, cod-liver oil, salmon oil, flaxseed oil, wheat germ oil, evening primrose oils, almond oil, babassu oil, borage oil, black currant seed oil, canola oil, castor oil, coconut oil, corn oil, cottonseed oil, emu oil, evening primrose oil, flax seed oil, grapeseed oil, groundnut oil (e.g., peanut), lanolin oil, linseed oil, mink oil, mustard seed oil, olive oil, palm oil, palm kernel oil, rapeseed oil, safflower oil, sesame oil, shark liver oil, silicon oil, soybean oil, sunflower oil, tree nut oil (e.g., almond, cashew, macadamia, walnut, hazelnut, pistachio, Brazil nuts, etc.), hydrogenated castor oil, hydrogenated coconut oil, hydrogenated palm oil, hydrogenated soybean oil, hydrogenated vegetable oil, a mixture of hydrogenated cottonseed oil and hydrogenated castor oil, partially hydrogenated soybean oil, a mixture of partially hydrogenated soybean oil and partially hydrogenated cottonseed oil, glyceryl trioleate, glyceryl trilinoleate, glyceryl trilinolenate, citrate thisocetyl triglyceride having 10-18 carbon atoms, a Ω3 polyunsaturated fatty acid triglyceride containing oil, omega-3 oil, omega-6 oil, and any combination thereof.

The percentage of the oil component of the composition ranges between 99% w/w and 10% w/w based on the weight of the composition, such as in between 80% w/w and 20% w/w, such as in between 60% w/w and 40% w/w. In preferred embodiment the percentage of non-toxic solvent ranges between 80% w/w and 50% w/w. The oil component of the compositions described herein may be a blend of more than one oil.

### Propylene Glycol

The compositions provided for herein are preferably substantially free of propylene glycol, more preferably propylene glycol-free. When propylene glycol is present it is present in an amount less than about 10% w/w.

Some concentrations of propylene glycol include from about 0.001% w/w to about 5% w/w, from about 0.005% w/w to about 5% w/w, from about 0.01% w/w to about 5% w/w, from about 0.05% w/w to about 5% w/w, from about 0.1% w/w to about 5% w/w, from about 0.5% w/w to about 5% w/w, from about 1% w/w to about 5% w/w, from about 1.5% w/w to about 5% w/w, from about 2% w/w to about 5% w/w, from about 2.5% w/w to about 5% w/w, from about 3% w/w to about 5% w/w, from about 3.5% w/w to about 5% w/w, from about 4% w/w to about 5% w/w, from about 4.5% w/w, from about 0.001% w/w to about 4.5% w/w, from about 0.005% w/w to about 4.5% w/w, from about 0.01% w/w to about 4.5% w/w, from about 0.05% w/w to about 4.5% w/w, from about 0.1% w/w to about 4.5% w/w, from about 0.5% w/w to about 4.5% w/w, from about 1% w/w to about 4.5% w/w, from about 1.5% w/w to about 4.5% w/w, from about 2% w/w to about 4.5% w/w, from about 2.5% w/w to about 4.5% w/w, from about 3% w/w to about 4.5% w/w, from about 3.5% w/w to about 4.5% w/w, from about 4% w/w to about 4.5% w/w, from about 0.001% w/w to about 4% w/w, from about 0.005% w/w to about 4% w/w, from about 0.01% w/w to about 4% w/w, from about 0.05% w/w to about 4% w/w, from about 0.1% w/w to about 4% w/w, from about 0.5% w/w to about 4% w/w, from about 1% w/w to about 4% w/w, from about 1.5% w/w to about 4% w/w, from about 2% w/w to about 4% w/w, from about 2.5% w/w to about 4% w/w, from about 3% w/w to about 4% w/w, from about 3.5% w/w to about 4% w/w, from about 0.001% w/w to about 3.5% w/w, from about 0.005% w/w to about 3.5% w/w, from about 0.01% w/w to about 3.5% w/w, from about 0.05% w/w to about 3.5% w/w, from about 0.1% w/w to about 3.5% w/w, from about 0.5% w/w to about 3.5% w/w, from about 1% w/w to about 3.5% w/w, from about 1.5% w/w to about 3.5% w/w, from about 2% w/w to about 3.5% w/w, from about 2.5% w/w to about 3.5% w/w, from about 3% w/w to about 3.5% w/w, from about 0.001% w/w to about 3% w/w, from about 0.005% w/w to about 3% w/w, from about 0.01% w/w to about 3% w/w, from about 0.05% w/w to about 3% w/w, from about 0.1% w/w to about 3% w/w, from about 0.5% w/w to about 3% w/w, from about 1% w/w to about 3% w/w, from about 1.5% w/w to about 3% w/w, from about 2% w/w to about 3% w/w, from about 2.5% w/w to about 3% w/w, from about 0.001% w/w to about 2.5% w/w, from about 0.005% w/w to about 2.5% w/w, from about 0.01% w/w to about 2.5% w/w, from about 0.05% w/w to about 2.5% w/w, from about 0.1% w/w to about 2.5% w/w, from about 0.5% w/w to about 2.5% w/w, from about 1% w/w to about 2.5% w/w, from about 1.5% w/w to about 2.5% w/w, from about 2% w/w to about 2.5% w/w, from about 0.001% w/w to about 2% w/w, from about 0.005% w/w to about 2% w/w, from about 0.01% w/w to about 2% w/w, from about 0.05% w/w to about 2% w/w, from about 0.1% w/w to about 2% w/w, from about 0.5% w/w to about 2% w/w, from about 1% w/w to about 2% w/w, from about 1.5% w/w to about 2% w/w, from about 0.001% w/w to about 1.5% w/w, from about 0.005% w/w to about 1.5% w/w, from about 0.01% w/w to about 1.5% w/w, from about 0.05% w/w to about 1.5% w/w, from about 0.1% w/w to about 1.5% w/w, from about 0.5% w/w to about 1.5% w/w, from about 1% w/w to about 1.5% w/w, from about 0.001% w/w to about 1% w/w, from about 0.005% w/w to about 1% w/w, from about 0.01% w/w to about 1% w/w, from about 0.05% w/w to about 1% w/w, from about 0.1% w/w to about 1% w/w, from about 0.5% w/w to about 1% w/w, from about 0.001% w/w to about 0.5% w/w, from about 0.005% w/w to about 0.5% w/w, from about 0.01% w/w to about 0.5% w/w, from about 0.05% w/w to about 0.5% w/w, from about 0.1% w/w to about 0.5% w/w, from about 0.001% w/w to about 0.1% w/w, from about 0.005% w/w to about 0.1% w/w, from about 0.01% w/w to about 0.1% w/w, from about 0.05% w/w to about 0.1% w/w, from about 0.001% w/w to about 0.05% w/w, from about 0.005% w/w to about 0.05% w/w, from about 0.01% w/w to about 0.05% w/w, or from about 0.001% w/w to about 0.005% w/w. In some embodiments, the propylene glycol is present at about 0.001% w/w, 0.005% w/w, 0.01% w/w, 0.05% w/w, 0.1% w/w, 0.5% w/w, 1% w/w, 1.5% w/w, 2% w/w, 2.5% w/w, 3% w/w, 3.5% w/w, 4% w/w, 4.5% w/w, or 5% w/w.

Propylene glycol may also be present in 5.5% w/w to about 10% w/w, from about 6% w/w to about 10% w/w, from about 6.5% w/w to about 10% w/w, from about 7% w/w to about 10% w/w, from about 7.5% w/w to about 10% w/w, from about 8% w/w to about 10% w/w, from about 8.5% w/w to about 10% w/w, from about 9% w/w to about 10% w/w, from about 9.5% w/w to about 10% w/w, from about 5% w/w to about 9.5% w/w, 5.5% w/w to about 9.5% w/w, from about 6% w/w to about 9.5% w/w, from about 6.5% w/w to about 9.5% w/w, from about 7% w/w to about 9.5% w/w, from about 7.5% w/w to about 9.5% w/w, from about 8% w/w to about 9.5% w/w, from about 8.5% w/w to about 9.5% w/w, from about 9% w/w to about 9.5% w/w, from about 5% w/w to about 9% w/w, 5.5% w/w to about 9% w/w, from about 6% w/w to about 9% w/w, from about 6.5% w/w to about 9% w/w, from about 7% w/w to about 9% w/w, from about 7.5% w/w to about 9% w/w, from about 8% w/w to about 9% w/w, from about 8.5% w/w to about 9% w/w, from about 5% w/w to about 8.5% w/w, 5.5% w/w to about 8.5% w/w, from about 6% w/w to about 8.5% w/w, from about 6.5% w/w to about 8.5% w/w, from about 7% w/w to about 8.5% w/w, from about 7.5% w/w to about 8.5% w/w, from about 8% w/w to about 8.5% w/w, from about 5% w/w to about 8% w/w, 5.5% w/w to about 8% w/w, from about 6% w/w to about 8% w/w, from about 6.5% w/w to about 8% w/w, from about 7% w/w to about 8% w/w, from about 7.5% w/w to about 8% w/w, from about 5% w/w to about 7.5% w/w, 5.5% w/w to about 7.5% w/w, from about 6% w/w to about 7.5% w/w, from about 6.5% w/w to about 7.5% w/w, from about 7% w/w to about 7.5% w/w, from about 5% w/w to about 7% w/w, 5.5% w/w to about 7% w/w, from about 6% w/w to about 7% w/w, from about 6.5% w/w to about 7% w/w, from about 5% w/w to about 6.5% w/w, 5.5% w/w to about 6.5% w/w, or from about 6% w/w to about 6.5% w/w. In some embodiments, propylene glycol is present at about 5% w/w, 5.5% w/w, 6% w/w, 6.5% w/w, 7% w/w, 7.5% w/w, 8% w/w, 8.5% w/w, 9% w/w, 9.5% w/w or 10% w/w.

### Vasodilator Concentrations

The concentration of the vasodilator, e.g., minoxidil or nitric oxide, in the present compositions may vary and may depend, for example, on the particular form of the composition, for example, whether the compositions are gelled compositions or non-gelled compositions. Broadly speaking, the minoxidil may be present in the present composition in an amount, which ranges from greater than about 0.01% w/w to about 8% w/w, and all combinations and subcombinations of ranges and specific amounts therein. As used herein, the term “%” refers to weight %, unless otherwise indicated. In addition, the total % of components in the present compositions may not exceed 100%. In preferred embodiments, the minoxidil may be present in a concentration of greater than about 3% w/w, with concentrations of greater than about 3% w/w to about 8% w/w being more preferred. In other preferred embodiments, concentrations of greater than 3% w/w are preferred, with concentrations of from greater than 3% w/w to about 8% w/w being more preferred. In the case of certain preferred compositions, including certain gel compositions, the minoxidil may be present in a concentration of from about 4% w/w to about 8% w/w, about 5% w/w to about 8% w/w, about 6% w/w to about 8% w/w and about 7% w/w to about 8% w/w. In even more preferred embodiments, the minoxidil may be present in an amount of from about 4% w/w to about 7% w/w, or about 5% w/w to about 6% w/w, with concentrations of about 5% w/w being particularly preferred.

Some concentrations of minoxidil include from about 0.001% w/w to about 5% w/w, from about 0.005% w/w to about 5% w/w, from about 0.01% w/w to about 5% w/w, from about 0.05% w/w to about 5% w/w, from about 0.1% w/w to about 5% w/w, from about 0.5% w/w to about 5% w/w, from about 1% w/w to about 5% w/w, from about 1.5% w/w to about 5% w/w, from about 2% w/w to about 5% w/w, from about 2.5% w/w to about 5% w/w, from about 3% w/w to about 5% w/w, from about 3.5% w/w to about 5% w/w, from about 4% w/w to about 5% w/w, from about 4.5% w/w, from about 0.001% w/w to about 4.5% w/w, from about 0.005% w/w to about 4.5% w/w, from about 0.01% w/w to about 4.5% w/w, from about 0.05% w/w to about 4.5% w/w, from about 0.1% w/w to about 4.5% w/w, from about 0.5% w/w to about 4.5% w/w, from about 1% w/w to about 4.5% w/w, from about 1.5% w/w to about 4.5% w/w, from about 2% w/w to about 4.5% w/w, from about 2.5% w/w to about 4.5% w/w, from about 3% w/w to about 4.5% w/w, from about 3.5% w/w to about 4.5% w/w, from about 4% w/w to about 4.5% w/w, from about 0.001% w/w to about 4% w/w, from about 0.005% w/w to about 4% w/w, from about 0.01% w/w to about 4% w/w, from about 0.05% w/w to about 4% w/w, from about 0.1% w/w to about 4% w/w, from about 0.5% w/w to about 4% w/w, from about 1% w/w to about 4% w/w, from about 1.5% w/w to about 4% w/w, from about 2% w/w to about 4% w/w, from about 2.5% w/w to about 4% w/w, from about 3% w/w to about 4% w/w, from about 3.5% w/w to about 4% w/w, from about 0.001% w/w to about 3.5% w/w, from about 0.005% w/w to about 3.5% w/w, from about 0.01% w/w to about 3.5% w/w, from about 0.05% w/w to about 3.5% w/w, from about 0.1% w/w to about 3.5% w/w, from about 0.5% w/w to about 3.5% w/w, from about 1% w/w to about 3.5% w/w, from about 1.5% w/w to about 3.5% w/w, from about 2% w/w to about 3.5% w/w, from about 2.5% w/w to about 3.5% w/w, from about 3% w/w to about 3.5% w/w, from about 0.001% w/w to about 3% w/w, from about 0.005% w/w to about 3% w/w, from about 0.01% w/w to about 3% w/w, from about 0.05% w/w to about 3% w/w, from about 0.1% w/w to about 3% w/w, from about 0.5% w/w to about 3% w/w, from about 1% w/w to about 3% w/w, from about 1.5% w/w to about 3% w/w, from about 2% w/w to about 3% w/w, from about 2.5% w/w to about 3% w/w, from about 0.001% w/w to about 2.5% w/w, from about 0.005% w/w to about 2.5% w/w, from about 0.01% w/w to about 2.5% w/w, from about 0.05% w/w to about 2.5% w/w, from about 0.1% w/w to about 2.5% w/w, from about 0.5% w/w to about 2.5% w/w, from about 1% w/w to about 2.5% w/w, from about 1.5% w/w to about 2.5% w/w, from about 2% w/w to about 2.5% w/w, from about 0.001% w/w to about 2% w/w, from about 0.005% w/w to about 2% w/w, from about 0.01% w/w to about 2% w/w, from about 0.05% w/w to about 2% w/w, from about 0.1% w/w to about 2% w/w, from about 0.5% w/w to about 2% w/w, from about 1% w/w to about 2% w/w, from about 1.5% w/w to about 2% w/w, from about 0.001% w/w to about 1.5% w/w, from about 0.005% w/w to about 1.5% w/w, from about 0.01% w/w to about 1.5% w/w, from about 0.05% w/w to about 1.5% w/w, from about 0.1% w/w to about 1.5% w/w, from about 0.5% w/w to about 1.5% w/w, from about 1% w/w to about 1.5% w/w, from about 0.001% w/w to about 1% w/w, from about 0.005% w/w to about 1% w/w, from about 0.01% w/w to about 1% w/w, from about 0.05% w/w to about 1% w/w, from about 0.1% w/w to about 1% w/w, from about 0.5% w/w to about 1% w/w, from about 0.001% w/w to about 0.5% w/w, from about 0.005% w/w to about 0.5% w/w, from about 0.01% w/w to about 0.5% w/w, from about 0.05% w/w to about 0.5% w/w, from about 0.1% w/w to about 0.5% w/w, from about 0.001% w/w to about 0.1% w/w, from about 0.005% w/w to about 0.1% w/w, from about 0.01% w/w to about 0.1% w/w, from about 0.05% w/w to about 0.1% w/w, from about 0.001% w/w to about 0.05% w/w, from about 0.005% w/w to about 0.05% w/w, from about 0.01% w/w to about 0.05% w/w, or from about 0.001% w/w to about 0.005% w/w. In some embodiments, the minoxidil is present at about 0.001% w/w, 0.005% w/w, 0.01% w/w, 0.05% w/w, 0.1% w/w, 0.5% w/w, 1% w/w, 1.5% w/w, 2% w/w, 2.5% w/w, 3% w/w, 3.5% w/w, 4% w/w, 4.5% w/w, or 5% w/w.

Minoxidil may also be present in 5.5% w/w to about 10% w/w, from about 6% w/w to about 10% w/w, from about 6.5% w/w to about 10% w/w, from about 7% w/w to about 10% w/w, from about 7.5% w/w to about 10% w/w, from about 8% w/w to about 10% w/w, from about 8.5% w/w to about 10% w/w, from about 9% w/w to about 10% w/w, from about 9.5% w/w to about 10% w/w, from about 5% w/w to about 9.5% w/w, 5.5% w/w to about 9.5% w/w, from about 6% w/w to about 9.5% w/w, from about 6.5% w/w to about 9.5% w/w, from about 7% w/w to about 9.5% w/w, from about 7.5% w/w to about 9.5% w/w, from about 8% w/w to about 9.5% w/w, from about 8.5% w/w to about 9.5% w/w, from about 9% w/w to about 9.5% w/w, from about 5% w/w to about 9% w/w, 5.5% w/w to about 9% w/w, from about 6% w/w to about 9% w/w, from about 6.5% w/w to about 9% w/w, from about 7% w/w to about 9% w/w, from about 7.5% w/w to about 9% w/w, from about 8% w/w to about 9% w/w, from about 8.5% w/w to about 9% w/w, from about 5% w/w to about 8.5% w/w, 5.5% w/w to about 8.5% w/w, from about 6% w/w to about 8.5% w/w, from about 6.5% w/w to about 8.5% w/w, from about 7% w/w to about 8.5% w/w, from about 7.5% w/w to about 8.5% w/w, from about 8% w/w to about 8.5% w/w, from about 5% w/w to about 8% w/w, 5.5% w/w to about 8% w/w, from about 6% w/w to about 8% w/w, from about 6.5% w/w to about 8% w/w, from about 7% w/w to about 8% w/w, from about 7.5% w/w to about 8% w/w, from about 5% w/w to about 7.5% w/w, 5.5% w/w to about 7.5% w/w, from about 6% w/w to about 7.5% w/w, from about 6.5% w/w to about 7.5% w/w, from about 7% w/w to about 7.5% w/w, from about 5% w/w to about 7% w/w, 5.5% w/w to about 7% w/w, from about 6% w/w to about 7% w/w, from about 6.5% w/w to about 7% w/w, from about 5% w/w to about 6.5% w/w, 5.5% w/w to about 6.5% w/w, or from about 6% w/w to about 6.5% w/w. In some embodiments, minoxidil is present at about 5% w/w, 5.5% w/w, 6% w/w, 6.5% w/w, 7% w/w, 7.5% w/w, 8% w/w, 8.5% w/w, 9% w/w, 9.5% w/w or 10% w/w. In some embodiments, minoxidil is present in an amount of about 11% w/w, 11.5% w/w, 12% w/w, 12.5% w/w, 13% w/w, 13.5% w/w, 14% w/w, 14.5% w/w, 15% w/w, 15.5% w/w, 16% w/w, 16.5% w/w, 17% w/w, 17.5% w/w, 18% w/w, 18.5% w/w, 19% w/w, 19.5% w/w or 20% w/w.

### Prostaglandin Analog Concentrations

Contemplated herein is the use of a prostaglandin analog for arresting or reversing hair loss, or both, and/or promoting hair growth. Bimatoprost, latanoprost, travoprost, unoprostone, fluprostenol and tafluprost are exemplary prostaglandin analogs.

The prostaglandin analog may be present at 0.1% w/w or 0.3% w/w. Other concentrations that the prostaglandin analog may be present are about 0.005% w/w to about 5% w/w, from about 0.01% w/w to about 5% w/w, from about 0.05% w/w to about 5% w/w, from about 0.1% w/w to about 5% w/w, from about 0.5% w/w to about 5% w/w, from about 1% w/w to about 5% w/w, from about 1.5% w/w to about 5% w/w, from about 2% w/w to about 5% w/w, from about 2.5% w/w to about 5% w/w, from about 3% w/w to about 5% w/w, from about 3.5% w/w to about 5% w/w, from about 4% w/w to about 5% w/w, from about 4.5% w/w, from about 0.001% w/w to about 4.5% w/w, from about 0.005% w/w to about 4.5% w/w, from about 0.01% w/w to about 4.5% w/w, from about 0.05% w/w to about 4.5% w/w, from about 0.1% w/w to about 4.5% w/w, from about 0.5% w/w to about 4.5% w/w, from about 1% w/w to about 4.5% w/w, from about 1.5% w/w to about 4.5% w/w, from about 2% w/w to about 4.5% w/w, from about 2.5% w/w to about 4.5% w/w, from about 3% w/w to about 4.5% w/w, from about 3.5% w/w to about 4.5% w/w, from about 4% w/w to about 4.5% w/w, from about 0.001% w/w to about 4% w/w, from about 0.005% w/w to about 4% w/w, from about 0.01% w/w to about 4% w/w, from about 0.05% w/w to about 4% w/w, from about 0.1% w/w to about 4% w/w, from about 0.5% w/w to about 4% w/w, from about 1% w/w to about 4% w/w, from about 1.5% w/w to about 4% w/w, from about 2% w/w to about 4% w/w, from about 2.5% w/w to about 4% w/w, from about 3% w/w to about 4% w/w, from about 3.5% w/w to about 4% w/w, from about 0.001% w/w to about 3.5% w/w, from about 0.005% w/w to about 3.5% w/w, from about 0.01% w/w to about 3.5% w/w, from about 0.05% w/w to about 3.5% w/w, from about 0.1% w/w to about 3.5% w/w, from about 0.5% w/w to about 3.5% w/w, from about 1% w/w to about 3.5% w/w, from about 1.5% w/w to about 3.5% w/w, from about 2% w/w to about 3.5% w/w, from about 2.5% w/w to about 3.5% w/w, from about 3% w/w to about 3.5% w/w, from about 0.001% w/w to about 3% w/w, from about 0.005% w/w to about 3% w/w, from about 0.01% w/w to about 3% w/w, from about 0.05% w/w to about 3% w/w, from about 0.1% w/w to about 3% w/w, from about 0.5% w/w to about 3% w/w, from about 1% w/w to about 3% w/w, from about 1.5% w/w to about 3% w/w, from about 2% w/w to about 3% w/w, from about 2.5% w/w to about 3% w/w, from about 0.001% w/w to about 2.5% w/w, from about 0.005% w/w to about 2.5% w/w, from about 0.01% w/w to about 2.5% w/w, from about 0.05% w/w to about 2.5% w/w, from about 0.1% w/w to about 2.5% w/w, from about 0.5% w/w to about 2.5% w/w, from about 1% w/w to about 2.5% w/w, from about 1.5% w/w to about 2.5% w/w, from about 2% w/w to about 2.5% w/w, from about 0.001% w/w to about 2% w/w, from about 0.005% w/w to about 2% w/w, from about 0.01% w/w to about 2% w/w, from about 0.05% w/w to about 2% w/w, from about 0.1% w/w to about 2% w/w, from about 0.5% w/w to about 2% w/w, from about 1% w/w to about 2% w/w, from about 1.5% w/w to about 2% w/w, from about 0.001% w/w to about 1.5% w/w, from about 0.005% w/w to about 1.5% w/w, from about 0.01% w/w to about 1.5% w/w, from about 0.05% w/w to about 1.5% w/w, from about 0.1% w/w to about 1.5% w/w, from about 0.5% w/w to about 1.5% w/w, from about 1% w/w to about 1.5% w/w, from about 0.001% w/w to about 1% w/w, from about 0.005% w/w to about 1% w/w, from about 0.01% w/w to about 1% w/w, from about 0.05% w/w to about 1% w/w, from about 0.1% w/w to about 1% w/w, from about 0.5% w/w to about 1% w/w, from about 0.001% w/w to about 0.5% w/w, from about 0.005% w/w to about 0.5% w/w, from about 0.01% w/w to about 0.5% w/w, from about 0.05% w/w to about 0.5% w/w, from about 0.1% w/w to about 0.5% w/w, from about 0.001% w/w to about 0.1% w/w, from about 0.005% w/w to about 0.1% w/w, from about 0.01% w/w to about 0.1% w/w, from about 0.05% w/w to about 0.1% w/w, from about 0.001% w/w to about 0.05% w/w, from about 0.005% w/w to about 0.05% w/w, from about 0.01% w/w to about 0.05% w/w, or from about 0.001% w/w to about 0.005% w/w. In some embodiments, the prostaglandin analog is present at about 0.001% w/w, 0.005% w/w, 0.01% w/w, 0.05% w/w, 0.1% w/w, 0.5% w/w, 1% w/w, 1.5% w/w, 2% w/w, 2.5% w/w, 3% w/w, 3.5% w/w, 4% w/w, 4.5% w/w, or 5% w/w. In some embodiments, the prostaglandin analog is present in an amount of about 11% w/w, 11.5% w/w, 12% w/w, 12.5% w/w, 13% w/w, 13.5% w/w, 14% w/w, 14.5% w/w, 15% w/w, 15.5% w/w, 16% w/w, 16.5% w/w, 17% w/w, 17.5% w/w, 18% w/w, 18.5% w/w, 19% w/w, 19.5% w/w or 20% w/w.

### Vitamin A Analog Concentrations

Tretinoin is all-trans-retinoic acid formed from the oxidation of the aldehyde group of retinene to a carboxyl group. Thus, structurally it is a vitamin A analog. It is highly reactive to light and moisture. Tretinoin is classified therapeutically as a keratolytic. The chemical name for tretinoin is: (all-E)-3,7-dimethyl-9-(2,6,6-trimethyl-1-cyclohexen-1-yl)-2,4,6,8-nonate-traenoic acid. The molecular formula is C₂₀H₂₈O₂ and molecular weight is 300.44. Vitamin A analogs include, but are not limited to, topical retinoids: tretinoin, alitretinoin, adapalene, and tazarotene.

The vitamin A analog is present in an amount of about 0.001% w/w to about 20% w/w relative to the total weight of the composition. In preferred embodiments the proportions are between about 0.001% and 20% by weight relative to the total weight of the composition. In more preferred embodiments the amount is selected from 0.025%, 0.05%, and 0.1% w/w.

Some concentrations of tretinoin include from about 0.001% w/w to about 5% w/w, from about 0.005% w/w to about 5% w/w, from about 0.01% w/w to about 5% w/w, from about 0.05% w/w to about 5% w/w, from about 0.1% w/w to about 5% w/w, from about 0.5% w/w to about 5% w/w, from about 1% w/w to about 5% w/w, from about 1.5% w/w to about 5% w/w, from about 2% w/w to about 5% w/w, from about 2.5% w/w to about 5% w/w, from about 3% w/w to about 5% w/w, from about 3.5% w/w to about 5% w/w, from about 4% w/w to about 5% w/w, from about 4.5% w/w, from about 0.001% w/w to about 4.5% w/w, from about 0.005% w/w to about 4.5% w/w, from about 0.01% w/w to about 4.5% w/w, from about 0.05% w/w to about 4.5% w/w, from about 0.1% w/w to about 4.5% w/w, from about 0.5% w/w to about 4.5% w/w, from about 1% w/w to about 4.5% w/w, from about 1.5% w/w to about 4.5% w/w, from about 2% w/w to about 4.5% w/w, from about 2.5% w/w to about 4.5% w/w, from about 3% w/w to about 4.5% w/w, from about 3.5% w/w to about 4.5% w/w, from about 4% w/w to about 4.5% w/w, from about 0.001% w/w to about 4% w/w, from about 0.005% w/w to about 4% w/w, from about 0.01% w/w to about 4% w/w, from about 0.05% w/w to about 4% w/w, from about 0.1% w/w to about 4% w/w, from about 0.5% w/w to about 4% w/w, from about 1% w/w to about 4% w/w, from about 1.5% w/w to about 4% w/w, from about 2% w/w to about 4% w/w, from about 2.5% w/w to about 4% w/w, from about 3% w/w to about 4% w/w, from about 3.5% w/w to about 4% w/w, from about 0.001% w/w to about 3.5% w/w, from about 0.005% w/w to about 3.5% w/w, from about 0.01% w/w to about 3.5% w/w, from about 0.05% w/w to about 3.5% w/w, from about 0.1% w/w to about 3.5% w/w, from about 0.5% w/w to about 3.5% w/w, from about 1% w/w to about 3.5% w/w, from about 1.5% w/w to about 3.5% w/w, from about 2% w/w to about 3.5% w/w, from about 2.5% w/w to about 3.5% w/w, from about 3% w/w to about 3.5% w/w, from about 0.001% w/w to about 3% w/w, from about 0.005% w/w to about 3% w/w, from about 0.01% w/w to about 3% w/w, from about 0.05% w/w to about 3% w/w, from about 0.1% w/w to about 3% w/w, from about 0.5% w/w to about 3% w/w, from about 1% w/w to about 3% w/w, from about 1.5% w/w to about 3% w/w, from about 2% w/w to about 3% w/w, from about 2.5% w/w to about 3% w/w, from about 0.001% w/w to about 2.5% w/w, from about 0.005% w/w to about 2.5% w/w, from about 0.01% w/w to about 2.5% w/w, from about 0.05% w/w to about 2.5% w/w, from about 0.1% w/w to about 2.5% w/w, from about 0.5% w/w to about 2.5% w/w, from about 1% w/w to about 2.5% w/w, from about 1.5% w/w to about 2.5% w/w, from about 2% w/w to about 2.5% w/w, from about 0.001% w/w to about 2% w/w, from about 0.005% w/w to about 2% w/w, from about 0.01% w/w to about 2% w/w, from about 0.05% w/w to about 2% w/w, from about 0.1% w/w to about 2% w/w, from about 0.5% w/w to about 2% w/w, from about 1% w/w to about 2% w/w, from about 1.5% w/w to about 2% w/w, from about 0.001% w/w to about 1.5% w/w, from about 0.005% w/w to about 1.5% w/w, from about 0.01% w/w to about 1.5% w/w, from about 0.05% w/w to about 1.5% w/w, from about 0.1% w/w to about 1.5% w/w, from about 0.5% w/w to about 1.5% w/w, from about 1% w/w to about 1.5% w/w, from about 0.001% w/w to about 1% w/w, from about 0.005% w/w to about 1% w/w, from about 0.01% w/w to about 1% w/w, from about 0.05% w/w to about 1% w/w, from about 0.1% w/w to about 1% w/w, from about 0.5% w/w to about 1% w/w, from about 0.001% w/w to about 0.5% w/w, from about 0.005% w/w to about 0.5% w/w, from about 0.01% w/w to about 0.5% w/w, from about 0.05% w/w to about 0.5% w/w, from about 0.1% w/w to about 0.5% w/w, from about 0.001% w/w to about 0.1% w/w, from about 0.005% w/w to about 0.1% w/w, from about 0.01% w/w to about 0.1% w/w, from about 0.05% w/w to about 0.1% w/w, from about 0.001% w/w to about 0.05% w/w, from about 0.005% w/w to about 0.05% w/w, from about 0.01% w/w to about 0.05% w/w, or from about 0.001% w/w to about 0.005% w/w. In some embodiments, the tretinoin is present at about 0.001% w/w, 0.005% w/w, 0.01% w/w, 0.05% w/w, 0.1% w/w, 0.5% w/w, 1% w/w, 1.5% w/w, 2% w/w, 2.5% w/w, 3% w/w, 3.5% w/w, 4% w/w, 4.5% w/w, or 5% w/w.

Tretinoin may also be present in 5.5% w/w to about 10% w/w, from about 6% w/w to about 10% w/w, from about 6.5% w/w to about 10% w/w, from about 7% w/w to about 10% w/w, from about 7.5% w/w to about 10% w/w, from about 8% w/w to about 10% w/w, from about 8.5% w/w to about 10% w/w, from about 9% w/w to about 10% w/w, from about 9.5% w/w to about 10% w/w, from about 5% w/w to about 9.5% w/w, 5.5% w/w to about 9.5% w/w, from about 6% w/w to about 9.5% w/w, from about 6.5% w/w to about 9.5% w/w, from about 7% w/w to about 9.5% w/w, from about 7.5% w/w to about 9.5% w/w, from about 8% w/w to about 9.5% w/w, from about 8.5% w/w to about 9.5% w/w, from about 9% w/w to about 9.5% w/w, from about 5% w/w to about 9% w/w, 5.5% w/w to about 9% w/w, from about 6% w/w to about 9% w/w, from about 6.5% w/w to about 9% w/w, from about 7% w/w to about 9% w/w, from about 7.5% w/w to about 9% w/w, from about 8% w/w to about 9% w/w, from about 8.5% w/w to about 9% w/w, from about 5% w/w to about 8.5% w/w, 5.5% w/w to about 8.5% w/w, from about 6% w/w to about 8.5% w/w, from about 6.5% w/w to about 8.5% w/w, from about 7% w/w to about 8.5% w/w, from about 7.5% w/w to about 8.5% w/w, from about 8% w/w to about 8.5% w/w, from about 5% w/w to about 8% w/w, 5.5% w/w to about 8% w/w, from about 6% w/w to about 8% w/w, from about 6.5% w/w to about 8% w/w, from about 7% w/w to about 8% w/w, from about 7.5% w/w to about 8% w/w, from about 5% w/w to about 7.5% w/w, 5.5% w/w to about 7.5% w/w, from about 6% w/w to about 7.5% w/w, from about 6.5% w/w to about 7.5% w/w, from about 7% w/w to about 7.5% w/w, from about 5% w/w to about 7% w/w, 5.5% w/w to about 7% w/w, from about 6% w/w to about 7% w/w, from about 6.5% w/w to about 7% w/w, from about 5% w/w to about 6.5% w/w, 5.5% w/w to about 6.5% w/w, or from about 6% w/w to about 6.5% w/w. In some embodiments, tretinoin is present at about 5% w/w, 5.5% w/w, 6% w/w, 6.5% w/w, 7% w/w, 7.5% w/w, 8% w/w, 8.5% w/w, 9% w/w, 9.5% w/w or 10% w/w. In some embodiments, tretinoin is present in an amount of about 11% w/w, 11.5% w/w, 12% w/w, 12.5% w/w, 13% w/w, 13.5% w/w, 14% w/w, 14.5% w/w, 15% w/w, 15.5% w/w, 16% w/w, 16.5% w/w, 17% w/w, 17.5% w/w, 18% w/w, 18.5% w/w, 19% w/w, 19.5% w/w or 20% w/w.

### 5-Alpha-Reductase Inhibitor Concentrations

Contemplated herein is the use of a 5-alpha-reductase inhibitor for arresting or reversing hair loss, or both, and/or promoting hair growth. Finasteride and Dutasteride are exemplary 5-alpha-reductase inhibitors.

Some concentrations of 5-alpha-reductase inhibitor include from about 0.001% w/w to about 5% w/w, from about 0.005% w/w to about 5% w/w, from about 0.01% w/w to about 5% w/w, from about 0.05% w/w to about 5% w/w, from about 0.1% w/w to about 5% w/w, from about 0.5% w/w to about 5% w/w, from about 1% w/w to about 5% w/w, from about 1.5% w/w to about 5% w/w, from about 2% w/w to about 5% w/w, from about 2.5% w/w to about 5% w/w, from about 3% w/w to about 5% w/w, from about 3.5% w/w to about 5% w/w, from about 4% w/w to about 5% w/w, from about 4.5% w/w, from about 0.001% w/w to about 4.5% w/w, from about 0.005% w/w to about 4.5% w/w, from about 0.01% w/w to about 4.5% w/w, from about 0.05% w/w to about 4.5% w/w, from about 0.1% w/w to about 4.5% w/w, from about 0.5% w/w to about 4.5% w/w, from about 1% w/w to about 4.5% w/w, from about 1.5% w/w to about 4.5% w/w, from about 2% w/w to about 4.5% w/w, from about 2.5% w/w to about 4.5% w/w, from about 3% w/w to about 4.5% w/w, from about 3.5% w/w to about 4.5% w/w, from about 4% w/w to about 4.5% w/w, from about 0.001% w/w to about 4% w/w, from about 0.005% w/w to about 4% w/w, from about 0.01% w/w to about 4% w/w, from about 0.05% w/w to about 4% w/w, from about 0.1% w/w to about 4% w/w, from about 0.5% w/w to about 4% w/w, from about 1% w/w to about 4% w/w, from about 1.5% w/w to about 4% w/w, from about 2% w/w to about 4% w/w, from about 2.5% w/w to about 4% w/w, from about 3% w/w to about 4% w/w, from about 3.5% w/w to about 4% w/w, from about 0.001% w/w to about 3.5% w/w, from about 0.005% w/w to about 3.5% w/w, from about 0.01% w/w to about 3.5% w/w, from about 0.05% w/w to about 3.5% w/w, from about 0.1% w/w to about 3.5% w/w, from about 0.5% w/w to about 3.5% w/w, from about 1% w/w to about 3.5% w/w, from about 1.5% w/w to about 3.5% w/w, from about 2% w/w to about 3.5% w/w, from about 2.5% w/w to about 3.5% w/w, from about 3% w/w to about 3.5% w/w, from about 0.001% w/w to about 3% w/w, from about 0.005% w/w to about 3% w/w, from about 0.01% w/w to about 3% w/w, from about 0.05% w/w to about 3% w/w, from about 0.1% w/w to about 3% w/w, from about 0.5% w/w to about 3% w/w, from about 1% w/w to about 3% w/w, from about 1.5% w/w to about 3% w/w, from about 2% w/w to about 3% w/w, from about 2.5% w/w to about 3% w/w, from about 0.001% w/w to about 2.5% w/w, from about 0.005% w/w to about 2.5% w/w, from about 0.01% w/w to about 2.5% w/w, from about 0.05% w/w to about 2.5% w/w, from about 0.1% w/w to about 2.5% w/w, from about 0.5% w/w to about 2.5% w/w, from about 1% w/w to about 2.5% w/w, from about 1.5% w/w to about 2.5% w/w, from about 2% w/w to about 2.5% w/w, from about 0.001% w/w to about 2% w/w, from about 0.005% w/w to about 2% w/w, from about 0.01% w/w to about 2% w/w, from about 0.05% w/w to about 2% w/w, from about 0.1% w/w to about 2% w/w, from about 0.5% w/w to about 2% w/w, from about 1% w/w to about 2% w/w, from about 1.5% w/w to about 2% w/w, from about 0.001% w/w to about 1.5% w/w, from about 0.005% w/w to about 1.5% w/w, from about 0.01% w/w to about 1.5% w/w, from about 0.05% w/w to about 1.5% w/w, from about 0.1% w/w to about 1.5% w/w, from about 0.5% w/w to about 1.5% w/w, from about 1% w/w to about 1.5% w/w, from about 0.001% w/w to about 1% w/w, from about 0.005% w/w to about 1% w/w, from about 0.01% w/w to about 1% w/w, from about 0.05% w/w to about 1% w/w, from about 0.1% w/w to about 1% w/w, from about 0.5% w/w to about 1% w/w, from about 0.001% w/w to about 0.5% w/w, from about 0.005% w/w to about 0.5% w/w, from about 0.01% w/w to about 0.5% w/w, from about 0.05% w/w to about 0.5% w/w, from about 0.1% w/w to about 0.5% w/w, from about 0.001% w/w to about 0.1% w/w, from about 0.005% w/w to about 0.1% w/w, from about 0.01% w/w to about 0.1% w/w, from about 0.05% w/w to about 0.1% w/w, from about 0.001% w/w to about 0.05% w/w, from about 0.005% w/w to about 0.05% w/w, from about 0.01% w/w to about 0.05% w/w, or from about 0.001% w/w to about 0.005% w/w. In some embodiments, the 5-alpha-reductase inhibitor is present at about 0.001% w/w, 0.005% w/w, 0.01% w/w, 0.05% w/w, 0.1% w/w, 0.5% w/w, 1% w/w, 1.5% w/w, 2% w/w, 2.5% w/w, 3% w/w, 3.5% w/w, 4% w/w, 4.5% w/w, or 5% w/w.

The 5-alpha-reductase inhibitor may also be present in 5.5% w/w to about 10% w/w, from about 6% w/w to about 10% w/w, from about 6.5% w/w to about 10% w/w, from about 7% w/w to about 10% w/w, from about 7.5% w/w to about 10% w/w, from about 8% w/w to about 10% w/w, from about 8.5% w/w to about 10% w/w, from about 9% w/w to about 10% w/w, from about 9.5% w/w to about 10% w/w, from about 5% w/w to about 9.5% w/w, 5.5% w/w to about 9.5% w/w, from about 6% w/w to about 9.5% w/w, from about 6.5% w/w to about 9.5% w/w, from about 7% w/w to about 9.5% w/w, from about 7.5% w/w to about 9.5% w/w, from about 8% w/w to about 9.5% w/w, from about 8.5% w/w to about 9.5% w/w, from about 9% w/w to about 9.5% w/w, from about 5% w/w to about 9% w/w, 5.5% w/w to about 9% w/w, from about 6% w/w to about 9% w/w, from about 6.5% w/w to about 9% w/w, from about 7% w/w to about 9% w/w, from about 7.5% w/w to about 9% w/w, from about 8% w/w to about 9% w/w, from about 8.5% w/w to about 9% w/w, from about 5% w/w to about 8.5% w/w, 5.5% w/w to about 8.5% w/w, from about 6% w/w to about 8.5% w/w, from about 6.5% w/w to about 8.5% w/w, from about 7% w/w to about 8.5% w/w, from about 7.5% w/w to about 8.5% w/w, from about 8% w/w to about 8.5% w/w, from about 5% w/w to about 8% w/w, 5.5% w/w to about 8% w/w, from about 6% w/w to about 8% w/w, from about 6.5% w/w to about 8% w/w, from about 7% w/w to about 8% w/w, from about 7.5% w/w to about 8% w/w, from about 5% w/w to about 7.5% w/w, 5.5% w/w to about 7.5% w/w, from about 6% w/w to about 7.5% w/w, from about 6.5% w/w to about 7.5% w/w, from about 7% w/w to about 7.5% w/w, from about 5% w/w to about 7% w/w, 5.5% w/w to about 7% w/w, from about 6% w/w to about 7% w/w, from about 6.5% w/w to about 7% w/w, from about 5% w/w to about 6.5% w/w, 5.5% w/w to about 6.5% w/w, or from about 6% w/w to about 6.5% w/w. In some embodiments, the 5-alpha-reductase inhibitor is present at about 5% w/w, 5.5% w/w, 6% w/w, 6.5% w/w, 7% w/w, 7.5% w/w, 8% w/w, 8.5% w/w, 9% w/w, 9.5% w/w or 10% w/w. In some embodiments, the 5-alpha-reductase inhibitor is present in an amount of about 11% w/w, 11.5% w/w, 12% w/w, 12.5% w/w, 13% w/w, 13.5% w/w, 14% w/w, 14.5% w/w, 15% w/w, 15.5% w/w, 16% w/w, 16.5% w/w, 17% w/w, 17.5% w/w, 18% w/w, 18.5% w/w, 19% w/w, 19.5% w/w or 20% w/w.

### Viscosity Agents

A viscosity agent is a substance that either increases or decreases the thickness of formulations provided for herein.

Suitable viscosity agents, which may be used include water soluble polymers, including anionic polymers and nonionic polymers. Useful polymers include vinyl polymers such as cross linked acrylic acid polymers with the CTFA name CARBOMER, pullulan, mannan, scleroglucans, polyvinylpyrrolidone, polyvinyl alcohol, guar gum, hydroxypropyl guar gum, xanthan gum, acacia gum, arabia gum, tragacanth, galactan, carob gum, karaya gum, locust bean gum, carrageenin, pectin, amylopectin, agar, quince seed (Cydonia oblonga Mill), starch (rice, corn, potato, wheat), algae colloids (algae extract), microbiological polymers such as dextran, succinoglucan, starch-based polymers such as carboxymethyl starch, methylhydroxypropyl starch, alginic acid-based polymers such as sodium alginate, alginic acid propylene glycol esters, acrylate polymers such as sodium polyacrylate, polyethylacrylate, polyacrylamide, polyethyleneimine, and inorganic water soluble materials such as bentonite, aluminum magnesium silicate, laponite, hectonite, and anhydrous silicic acid. Combinations of the foregoing may also be used in embodiments. In some embodiments, a carbomer such as homopolymers, copolymers, and interpolymers may be added as a viscosity agent to control the rheological properties of the cream or ointment formulas and add stability to formulations.

Also contemplated are nonaqueous viscosity agents such as polyisobutene, hydrogenated polyisobutene, trilaurin, triarachidin, tribehenin, tricaprin, tricaprylin, trierucin, triheptanoin, triheptylundecanoin, triisononanoin, triisopalmitin, triisostearin, trilinolein, trimyristin, trioctanoin, triolein, tripalmitin, tripalmitolein, triricinolein, tristearin, triundecanoin, glyceryl triacetyl hydroxystearate, glyceryl triacetyl ricinoleate, glyceryl stearate diacetate, octyldodecyl stearoyl stearate, high molecular weight silicones and the like.

Where utilized, a viscosity agent may be present in an amount from about 0.1% to about 5% by weight of the composition, in some embodiments from about 0.25% to about 1% by weight of the composition, in some embodiments from about 0.1% to about 0.6% by weight of the composition.

The amount of thickening agent employed in the present compositions may vary and depends, for example, on the particular polymer and solvent employed, the quantity of therapeutic agent, the desired viscosity of the final composition and the like. Generally speaking, the thickening agent may be employed in an amount to provide the compositions with a desired viscosity. Preferably, the thickening agent may be employed in an amount which ranges from about 0.01% to about 50%, and all combinations and subcombinations of ranges and specific amounts therein. More preferably, the thickening agent may be employed in an amount of from about 0.1% to about 3%, with from about 0.15% to about 0.6% being even more preferred.

### Other Components

In some embodiments, the composition of the present invention can be admixed with other carrier substances known in the art. Examples of carrier substances include but not limited to glycerine, aloe vera gel, allantoin, vitamin A and E, PPG2 myristyl propionate, and the like. The said mixture of composition and carrier substance can be administered topically in the form of solutions, creams, gels, lotions, shampoo, paste (See, e.g, Rasmusson etal., EP 0285 382, 1988).

The compositions disclosed herein may include one or more additional components. Such additional components include but are not limited to anti-static agents, buffering agents, bulking agents, conservational agents, chelating agents, cleansers, colorants, conditioners, deodorants, diluents, dyes, fragrances, hair conditioners, pearlescent aids, perfuming agents, permeation enhancers, pH-adjusting agents, preservatives, protectants, skin penetration enhancers, softeners, solubilizers, ionization agents, and antioxidants like flavonoids and phenolics. As is known to one skilled in the art, in some instances a specific additional component may have more than one activity, function or effect.

In some embodiments, the additional component is a pH adjusting agent or a buffering agent. Suitable buffering agents include but are not limited to acetic acid, adipic acid, calcium hydroxide, citric acid, glycine, hydrochloric acid, lactic acid, magnesium aluminometasilicates, phosphoric acid, sodium carbonate, sodium citrate, sodium hydroxide, sorbic acid, succinic acid, tartaric acid, and derivatives, salts and mixtures thereof.

### Method of Manufacture

An example of a typical process is as follows: An appropriate amount of the therapeutic agent is dissolved in an appropriate pharmaceutically acceptable solvent. To further increase the load of the therapeutic agent or to facilitate the process, the therapeutic agent can be solubilized in an appropriate solvent or mixture of solvents prior to adding therapeutic agent to the oil. The solvents used in such processes can then be partially or completely removed, depending on the tolerance of the formulation to the residual solvents.

A wide variety of methods may be used for preparing the compositions of the present invention. Broadly speaking, the compositions may be prepared by combining together the components of the compositions, as described herein, at a temperature and for a time sufficient to preferably provide an oil-based composition. The term “combining together”, as used herein, means that all of the components of the compositions may be combined and mixed together at about the same time. For example, the compositions may be prepared by combining together the therapeutic agent, thickening agent, pharmaceutically acceptable solvent and water to provide the non-gelled composition. In certain preferred embodiments, the term “combining together” means that the various components may be combined in one or more preferential sequences to provide the desired product.

In methods for preparing the compositions, the water and thickening agent may preferably be combined together to produce a uniformly dispersed and hydrated mixture. To this can be added the solvent may be added. The therapeutic agent may then be added to the mixture, which may then be blended until the therapeutic agent is dissolved. Alternatively, the therapeutic agent may be first dissolved in the solvent, and then this mixture may be added to the mixture of the thickening agent and water.

In addition, a composition of the present invention, including gels comprising from greater than 0.01% w/w to about 20% w/w therapeutic agent(s), from about 10% w/w to about 50% w/w of an alcohol, from about 50% w/w to about 99% w/w of at least one oil, from about 0.01% w/w to about 3% w/w of a viscosity agent, and from about 0% w/w to about 40% w/w water can be conveniently and effectively produced by the methods disclosed herein. In preferred form, the method may comprise providing a solution comprising a therapeutic agent, an oil and a portion of the alcohol (step a). The method further preferably comprises providing a dispersion comprising the thickening agent, the remaining portion of the alcohol, and the water (step b). Both the solution and dispersion may be obtained, for example, by combining together the various components of the solution and dispersion, respectively. The solution and the dispersion may then be combined until a uniform composition is produced (step c).

Depending upon the thickening agent chosen, it may be possible to add all of the alcohol to the dispersion of step (b), rather than adding a portion of the alcohol to the therapeutic agent solution of step (a). Some thickening agents may not tolerate the high alcohol/low water environment, however, or will not produce a satisfactory or workable dispersion. Additionally, it has been found that the addition of some of the alcohol to the solution of step (a) may promote dissolution of the therapeutic agent, and may, for example, avoid the necessity of heating the solution and permit step (a) to be performed at room temperature. Thus it is preferable that about 50% of the alcohol is used in the solution of step (a), and the remaining 50% is used in the dispersion of step (b).

### Methods of Use

The present invention further provides a method of treating, alleviating or preventing a hair loss disorder, comprising administering a therapeutically effective amount of the compositions described herein to an area of skin affected by hair loss. The method may comprise topical treatment as a single application, or it may comprise periodic treatment over an extended treatment time period as needed.

Typically, the composition may be applied repeatedly for a sustained period of time topically on the part of the body to be treated, for example, the scalp. The dosage regimen will generally involve regular, such as daily or twice daily, administration for a period of treatment of at least one month, or at least three months, or at least six months.

Alternatively, the composition may be applied intermittently, or in a pulsed manner. Accordingly, an alternative embodiment of the invention is to apply the composition on an intermittent or pulsed dosage schedule. For example, the composition of the invention may be used for two or more days, stopped, then restarted again at a time from between a few days, 2 weeks to 3 months later, and at even more long-spaced intervals in the case of the scalp.

For topical use on the skin and/or the scalp, the composition can be advantageously formulated using ointments, creams, gels, solutions or lotions as a carrier of the active ingredient. Also, these formulations may or may not contain preservatives, depending on the dispenser and nature of use. Such preservatives include those mentioned above, and methyl-, propyl-, or butyl-parahydroxybenzoic acid, betain, chlorhexidine, benzalkonium chloride, and the like. Typically, the dose to be applied is in the range of about 0.1 ng to about 100 mg per day, or about 1 ng to about 10 mg per day, or about 10 ng to about 1 mg per day depending on the hair growth stimulant, and/or hair growth stimulating agent and/or hair density increasing agent and/or hair loss prevention agent and the formulation. To achieve the daily amount of medication depending on the formulation, the therapeutic agent may be administered at least once a day but may be applied several times daily.

### Utility

The compositions of the present invention may be advantageously employed to treat and/or prevent a region of hair loss or alopecia in a patient, e.g., human. Generally speaking, the methods may comprise topically administering to the region a composition as described herein. The life of a hair is subjected to a cycle, called the pilar cycle, during which the hair grows (anagen), transitions (catagen), and falls out (telogen), before being replaced by a new hair which appears in the same follicle and the cycle is repeated. This constant renewal process undergoes a natural change during aging. The hair cycles become shorter, resulting in finer, shorter hairs. Hair loss results when this process is accelerated or disturbed, i.e., the growth phases become shorter, the passage of hair into the telogen phase is earlier and hairs fall out in larger numbers. Successive shortening growth cycles may result in increasingly fine and short hair, which is slowly converted into fluff. This phenomenon may lead to progressive hair thinning and may eventually lead to baldness.

Dermatologists recognize many different types of hair loss, the most common by far being androgenetic alopecia (also known as “pattern baldness”), wherein humans begin losing scalp hair as they get older. While this type of hair loss is more common in males, it also occurs in women. This type of alopecia may be characterized by progressive thinning, as discussed above, or may be characterized by hair loss with little diffuse hair thinning, such as frontal hair loss, mid-anterior balding, bitemporal recession, and/or vertex balding. Alopecia areata, anagen hair loss, traction alopecia and diffuse alopecia, such as telogen effluvium are other presentations of hair loss, which may be distinguished from androgenetic alopecia. These other forms or hair loss may also be treated with topical therapeutic agent as described herein.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### CITATION LIST

### Patent Literature

U.S. Pat. No. 4,139,619—Chidsey, III (1979)

U.S. Pat. No. 4,377,584—Rasmusson et al. (1983)

U.S. Pat. No. 4,828,837—Uster et al. (1989)

U.S. Pat. No. 5,030,442—Uster et al. (1991)

U.S. Pat. No. 5,225,189—Pena (1993)

U.S. Pat. No. 5,407,944—Goldman (1995)

U.S. Pat. No. 5,480,889—Goldman (1996)

U.S. Pat. No. 5,578,599—Diani et al. (1996)

U.S. Pat. No. 5,620,980—Samour (1997)

U.S. Pat. No. 5,643,942—Hester, Jr. et al. (1997)

U.S. Pat. No. 5,834,014—Weiner et al. (1998)

U.S. Pat. No. 5,981,543—Gormley et al. (1999)

U.S. Pat. No. 6,187,815—Hallam et al. (2001)

U.S. Pat. No. 6,255,313—Suzuki et al. (2001)

U.S. Pat. No. 6,262,105—Johnstone et al. (2001)

U.S. Pat. No. 6,265,412—Kimura et al. (2001)

U.S. Pat. No. 6,376,557—Zaveri (2002)

U.S. Pat. No. 6,465,514—Hallam et al. (2002)

U.S. Pat. No. 6,946,120—Wal-Chiu So et al. (2005)

U.S. Pat. No. 7,388,029—deLong et al. (2008)

U.S. Pat. No. 7,407,987—deLong et al. (2008)

U.S. Pat. No. 7,442,369—Pena et al. (2008)

U.S. Pat. No. 7,749,489—Malek (2010)

WO1988001502—Hatzenbuhler et al. (1988)

WO2000050007—Patel et al. (2000)

WO2001028555—Feng-Jing et al. (2001)

WO2002011698—Pena et al. (2002)

WO2008116135—McCook et al. (2008)

WO2008117117—Trigiante (2008)

WO2009040818—Temtsin (2009)

WO2009098595—Tamarkin et al. (2009)

WO2009151828—Cohen et al. (2009)

WO2010036947—Ali et al. (2010)

WO2011082235—Miller et al. (2011)

WO2012078649—Barman et al. (2012)

WO2012106249—Woodward et al. (2012)

### Non-Patent Literature

Wolf, et al., 2003 Dermatology Online Journal 9(3): 7

1. A topical oil-based pharmaceutical composition for slowing or stopping hair loss and/or promoting hair regrowth, said composition comprising:
(a) at least one therapeutic agent selected from a vasodilator, a prostaglandin analog, a 5-alpha-reductase inhibitor and a vitamin A analog;
(b) at least one oil;
(c) a viscosity agent;
(d) water, and
(e) alcohol,
wherein said composition is propylene glycol-free and does not reverse the affect of chemically or thermally straightened hair.

2. A topical oil-based pharmaceutical composition for slowing or stopping hair loss and/or promoting hair regrowth, said composition comprising:
(a) at least one therapeutic agent selected from a vasodilator, a prostaglandin analog, a 5-alpha-reductase inhibitor and a vitamin A analog;
(b) at least one oil;
(c) a viscosity agent;
(d) water, and
(e) alcohol,
wherein said composition is substantially free of propylene glycol and does not reverse the affect of chemically or thermally straightened hair.

3. The composition of claim 1 or 2, wherein the therapeutic agent is present in an amount of about 0.01% w/w to about 20% w/w when it is a vasodilator, about 0.01% w/w to about 20% w/w when it is a prostaglandin analog, about 0.01% w/w to about 20% w/w when it is a 5-alpha-reductase inhibitor and/or about 0.01% w/w to about 20% w/w when it is a vitamin A analog.

4. The composition of claim 1, wherein the at least one therapeutic agent is a combination selected from a vasodilator and a prostaglandin analog, a vasodilator and a 5-alpha-reductase inhibitor, a vasodilator and a vitamin A analog, a prostaglandin analog and a 5-alpha-reductase inhibitor, a prostaglandin analog and a vitamin A analog, and a 5-alpha-reductase inhibitor and a vitamin A analog.

5. The composition of claim 1, wherein the at least one therapeutic agent is a combination selected from a vasodilator, a prostaglandin analog, and a 5-alpha-reductase inhibitor; a vasodilator, a prostaglandin analog and a vitamin A analog; a prostaglandin analog, a 5-alpha-reductase inhibitor and a vitamin A analog; and a vasodilator, a 5-alpha-reductase inhibitor and a vitamin A analog.

6. The composition of claim 1, wherein the vasodilator is minoxidil.

7. The composition of claim 6, wherein the minoxidil is present in an amount of about 2% to about 10%.

8. The composition of claim 1, wherein the prostaglandin analog is bimatoprost.

9. The composition of claim 8, wherein the bimatoprost is present in an amount of about 0.03% to about 5% by weight of the composition.

10. The composition of claim 1, wherein the 5-alpha-reductase inhibitor is selected from finasteride and dutasteride.

11. The composition of claim 10, wherein the finasteride is present in an amount of about 0.25% to about 5% by weight of the composition.

12. The composition of claim 10, wherein the 5-alpha-reductase inhibitor is dutasteride.

13. The composition of claim 10, wherein the 5-alpha-reductase inhibitor is finasteride.

14. The composition of claim 1, wherein the vitamin A analog is tretinoin.

15. The composition of claim 14, wherein the tretinoin is present in an amount of about 0.025% to about 0.1% by weight of the composition.

16. The composition of claim 1, wherein the oil originates from vegetable, marine or animal sources or is a synthetic oil.

17. The composition of claim 1, wherein the oil is a saturated, unsaturated or polyunsaturated oil.

18. The composition of claim 1, wherein the oil is selected from borage seed oil, syzigium aromaticum oil, hempseed oil, herring oil, cod-liver oil, salmon oil, flaxseed oil, wheat germ oil, evening primrose oils, almond oil, babassu oil, borage oil, black currant seed oil, canola oil, castor oil, coconut oil, corn oil, cottonseed oil, emu oil, evening primrose oil, flax seed oil, grapeseed oil, groundnut oil (e.g., peanut), lanolin oil, linseed oil, mink oil, mustard seed oil, olive oil, palm oil, palm kernel oil, rapeseed oil, safflower oil, sesame oil, shark liver oil, silicon oil, soybean oil, sunflower oil, tree nut oil (e.g., almond, cashew, macadamia, walnut, hazelnut, pistachio, Brazil nuts, etc.), hydrogenated castor oil, hydrogenated coconut oil, hydrogenated palm oil, hydrogenated soybean oil, hydrogenated vegetable oil, a mixture of hydrogenated cottonseed oil and hydrogenated castor oil, partially hydrogenated soybean oil, a mixture of partially hydrogenated soybean oil and partially hydrogenated cottonseed oil, glyceryl trioleate, glyceryl trilinoleate, glyceryl trilinolenate, citrate thisocetyl triglyceride having 10-18 carbon atoms, a Ω3 polyunsaturated fatty acid triglyceride containing oil, omega-3 oil, omega-6 oil, and any combination thereof.

19. The composition of claim 1, wherein the oil is present in an amount of between about 50% to about 99%.

20. The composition of claim 1, wherein the viscosity agent is a carbomer.

21. The composition of claim 1, wherein the viscosity agent is present in an amount of between about 0.1% to about 5%.

22. The composition of claim 1, wherein further comprising pharmaceutically acceptable additives and ingredients selected from the group consisting of hair conditioners, panthenol derivatives, calcium pantothenate, colorants, fragrances, fragrance modifiers, vitamin E, penetration modifiers, surfactants, cosmetic agents, fatty acids and fatty acid esters, herbal extracts, henna, emulsifiers, wetting agents, sunscreens, and anti-irritants.

23. The composition of claim 1, wherein the water is present in an amount of less than about 30% w/w of the final composition.

24. The composition of claim 1, wherein the alcohol is present in an amount of between 0.1% w/w and about 50% w/w of the final composition.

25. The composition of claim 1, wherein the composition is an oil, cream, ointment, emulsion, gel, or lotion.

26. A method for treating or preventing hair loss in a region of a patient, said method comprising topically administering to said region a composition according to claim 1.

27. A method of claim 26, wherein said hair loss comprises androgenetic alopecia, frontal hair loss, bitemporal recession, vertex balding, mid-anterior balding, alopecia areata, anagen hair loss, diffuse alopecia, telogen effluvium, and fraction alopecia.

28. The method of claim 26, wherein the composition is administered on the skin once or twice a day.

29. The method of claim 26, wherein the composition is administered for at least a week.

30. The method of claim 26, wherein the composition is applied to the scalp.

31. A method of preparing a topical oil-based composition, said method comprising, based on the weight of the final composition:
(a) from about 0.001% w/w to about 20% w/w of at least one therapeutic agent;
(b) from about 50% w/w to about 99% w/w of an oil;
(c) from about 10% w/w to about 50% w/w of an alcohol
(d) from about 0.01% w/w to about 50% w/w of a viscosity agent; and
(e) from about 0% w/w to about 30% w/w water.
wherein said therapeutic agent is substantially solubilized in said composition, said process comprising:
(1) providing a solution comprising said therapeutic agent, said oil, and a portion of said alcohol;
(2) providing a dispersion comprising said viscosity agent, the remaining portion of said alcohol, and said water; and
(3) combining said solution and said dispersion to provide a final composition.
